(19) United States
(12) Patent Application Publication
(10) Pub. No.: US 2025/0195053 A1
(43) Pub. Date: Jun. 19, 2025

(54) STERILE INTERFACE FOR ARTICULATED SURGICAL INSTRUMENTS

(71) Applicant: Distalmotion SA, Epalinges (CH)

(72) Inventor: Ricardo Daniel Rita BEIRA, Lausanne (CH)

(21) Appl. No.: 19/070,318

(22) Filed: Mar. 4, 2025

Related U.S. Application Data
(63) Continuation of application No. 18/151,376, filed on Jan. 6, 2023, now Pat. No. 12,262,880.
Application No. 18/151,376 is a continuation of application No. 17/351,118, filed on Jun. 17, 2021, now Pat. No. 11,571,195.
Application No. 17/351,118 is a division of application No. 15/536,573, filed on Jun. 15, 2017, now Pat. No. 11,039,820.
(60) Provisional application No. 62/094,079, filed on Dec. 19, 2014.

(51) Int. Cl. A61B 17/00; A61B 34/00; A61B 34/30; A61B 34/35; A61B 46/10; A61B 90/00; B25J 19/00

(52) U.S. Cl. CPC A61B 17/00234; A61B 34/30; A61B 34/35; A61B 34/70; A61B 46/10; B25J 19/0075; A61B 2017/00477; A61B 2034/302; A61B 2090/0813

(57) ABSTRACT

A sterile interface for a surgical platform is provided, optionally to be used with a mechanical telemanipulator. The sterile interface is configured to allow for transmission of motion without dimensional inconsistencies between a non-sterile surgical platform and a sterile surgical instrument that are related to one another in a master-slave configuration. The sterile interface is configured to allow for multiple changes of sterile surgical instruments during a surgical procedure without contaminating the sterile field. The sterile interface allows for interchangeable sterile articulated surgical instruments to be attached to the surgical platform without coming into contact with non-sterile portions of the surgical platform.

### CROSS-REFERENCE TO RELATED APPLICATIONS

This application is a divisional of U.S. patent application Ser. No. 15/536,573, filed Jun. 15, 2017, now U.S. Pat. No. 11,039,820, which is a national phase of International PCT Patent Application Serial No. PCT/IB2015/002487, filed Dec. 18, 2015, which claims the benefit of U.S. Provisional Patent Application Ser. No. 62/094,079, filed Dec. 19, 2014, the entire contents of each of which are incorporated herein by reference.

### FIELD OF THE INVENTION

A sterile interface for surgical instruments is provided. More particularly, a sterile interface is provided whereby a sterile instrument portion is attached or detached from a surgical device platform that is not in the sterile field. Even more particularly, the present invention relates to a sterile interface wherein articulated surgical instruments, which may be laparoscopic instruments, may be attached or detached from a surgical platform. The sterile interface allows for the rapid, easy, attachment and detachment of sterile articulated surgical instruments from a surgical platform several times during a surgical procedure, thus allowing the operator to use a multitude of surgical instruments during one procedure while maintaining a sterile surgical field, but while also not requiring the sterilization of the entire surgical platform.

### BACKGROUND OF THE INVENTION

Open surgery is still the standard technique for most surgical procedures. It has been used by the medical community for several decades and consists of performing the surgical tasks by a long incision in the abdomen or other body cavity, through which traditional surgical tools are inserted. However, due to the long incision, this approach is extremely invasive for the patient, resulting in substantial blood loos during the surgery and long and painful recovery periods in an in-patient setting.

In order to reduce the invasiveness of open surgery, laparoscopy, a minimally invasive technique, was developed. Instead of a single long incision, one or more smaller incisions are made in the patient through which appropriately sized surgical instruments and endoscopic cameras are inserted. Because of the low degree of invasiveness, laparoscopic techniques reduce blood loss and pain while also shortening hospital stays. When performed by experienced surgeons, these techniques can attain clinical outcomes similar to open surgery. However, despite the above-mentioned advantages, laparoscopy requires advanced surgical skills to manipulate the generally rigid and long instrumentation through small incisions in the patient.

Traditionally, laparoscopic instruments, such as graspers, dissectors, scissors and other tools, have been mounted on straight shafts. These shafts are inserted through small incisions into the patient's body and, because of that, their range of motion inside the body is reduced. The entry incision acts as a point of rotation, decreasing the surgeon's freedom for positioning and orientating the instruments inside the patient. Therefore, due to the drawbacks of currently available instrumentation, laparoscopic procedures are mainly limited to use in simple surgeries, while only a small minority of surgeons is able to use them in complex procedures.

Laparoscopic instruments can be provided as disposable or reusable medical devices. Disposable devices are thrown away after each utilization, without having the need to be cleaned. On the other hand, reusable devices must be cleaned and sterilized after each procedure. In many instances, cost-effectiveness and operating room efficiency requires that instruments be cleaned, sterilized and re-used.

Several laparoscopic instruments may be used during a single surgical procedure. For example, graspers, dissectors and scissors may all need to be used. The present Applicants have demonstrated the use of articulated laparoscopic surgical instruments in conjunction with a mechanical telemanipulator, which allows the surgeon to have control over the instruments with a master-slave configuration based upon mechanical transmission of the surgeon's hand movements to the surgical instruments at pre-determined levels of amplification.

In this context, and in the context of other remotely actuated instrument systems, it is often desirable to detach and attach multiple instruments during a single procedure or period of operation. Particularly in the surgical context, although also when working in delicate, sensitive or contaminated environments, it is often desirable to create a sterile interface wherein the instruments being attached and detached are sterile but the platform to which they are attached is not in the sterile field.

Prior examples of detachable sterile surgical instruments are known, but they have functional or dimensional drawbacks. In any remotely actuated system, the interface between sterile and non-sterile components must not only be designed in such a way as to maintain the sterility of, for example, the surgical instruments, but it must also provide a faithful transmission of motion from the remote actuator to the distally located instruments. Thus, each degree of freedom provided to the user of the remotely actuated system must be reproduced through transmission elements at the junction between the detachable instrument and the platform without dimensional inaccuracies or backlash. In addition, the connector element is often a single use or limited use product and so manufacturing costs should be relatively cheap. Prior interfaces, such as those shown in U.S. Pat. No. 7,699,855, have these known drawbacks due to their design elements, which typically transmit motion through reduced diameters and, thus, are susceptible to inaccuracies, backlash, other unwanted movements and incomplete transmission of motion. Prior interfaces, such as those found in U.S. Pat. No. 7,699,855 are limited-use and can only be taken through a certain number of sterilization cycles before becoming inoperative when connected with the surgical platform.

Accordingly, an aim of the present invention is to overcome the aforementioned drawbacks of known devices by providing a sterile interface for remotely actuated surgical devices wherein sterile surgical instruments can be easily attached and detached from a non-sterile surgical platform. An additional aim is for the interface to provide faithful transmission of motion from the remote, non-sterile platform to the distally located sterile surgical instruments without dimensional inaccuracies or backlash. An additional aim is to provide single use interface elements that are inexpensive to manufacture but that nevertheless have tolerances that provide for the aforementioned faithful transmission of motion. An alternative aim is to provide interface elements that are relatively inexpensive to manufacture but are designed to be taken through multiple sterilization cycles without needing to be replaced, thus reducing overall operating room costs.

### SUMMARY OF THE INVENTION

These aims and other advantages are realized in a new sterile interface for the attachment of sterile surgical instruments to a non-sterile surgical platform. The sterile interface is intended to be used with articulated surgical instruments that are attached to a surgical platform. The surgical platform can be provided in the context of a mechanical telemanipulator.

In various embodiments, the sterile interface can be used in connection with a mechanical telemanipulator with a master-slave architecture and a mechanical transmission system. This enables a natural replication of user hand movements on a proximal handle at end-effector elements.

The sterile interface is designed such that surgical instruments, and in particular embodiments, laparoscopic surgical instruments, can be attached and detached from the mechanical surgical platform several times during a single surgical procedure. The sterile interface of the present invention is designed in such a way that sterilization is possible, allowing for several cycles of use before the interface elements need to be replaced.

### BRIEF DESCRIPTION OF THE FIGURES

FIG. 1 shows a mechanical telemanipulator with a detachable surgical instrument according to an embodiment of the present invention.

FIG. 2 shows the kinematics of a mechanical telemanipulator with a detachable surgical instrument according to an embodiment of the present invention.

FIG. 3 shows a surgical instrument detached from a mechanical telemanipulator according to an embodiment of the present invention.

FIG. 4 shows the kinematics associated with a surgical instrument detached from a mechanical telemanipulator according to an embodiment of the present invention.

FIG. 5 shows a detachable surgical instrument according to an embodiment of the present invention.

FIGS. 6 through 11 show various articulated end-effector links in various positions according to various embodiments of the present invention.

FIG. 12 shows the rotational elements of an interface portion of a surgical instrument according to an embodiment of the present invention.

FIG. 13 shows the rotational kinematics of an interface portion of a surgical instrument according to an embodiment of the present invention.

FIG. 14 shows a schematic view of the rotational elements of an interface portion of a surgical instrument according to an embodiment of the present invention.

FIGS. 15 and 16 show the mechanical transmission elements of a mechanical telemanipulator in conjunction with a detached surgical instrument according to an embodiment of the present invention.

FIG. 17 through 21 show various perspective views of an interface element in accordance with various embodiments of the present invention.

FIGS. 22 and 23 show schematic views of kinematics associated with a mechanical telemanipulator according to an embodiment of the present invention.

FIGS. 24 and 25 show perspective views of the attachment of elements of a surgical instrument to a surgical platform according to an embodiment of the present invention.

FIG. 26 shows a perspective view of various elements of a fixation ring for attachment of a sterile cover according to an embodiment of the present invention.

### DETAILED DESCRIPTION OF THE INVENTION

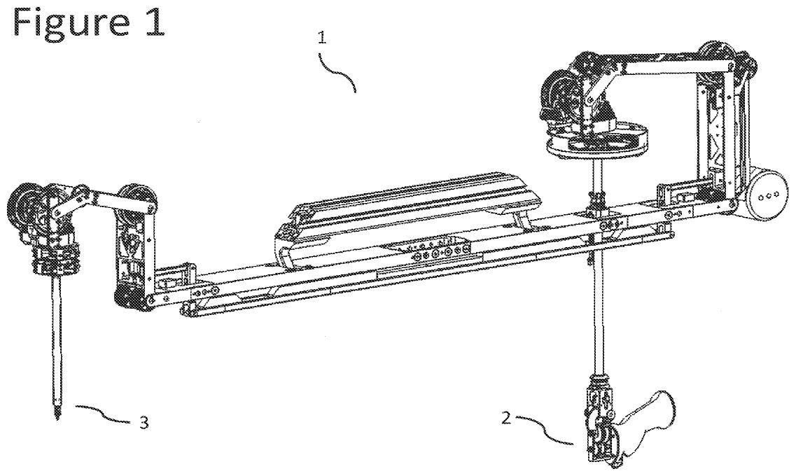

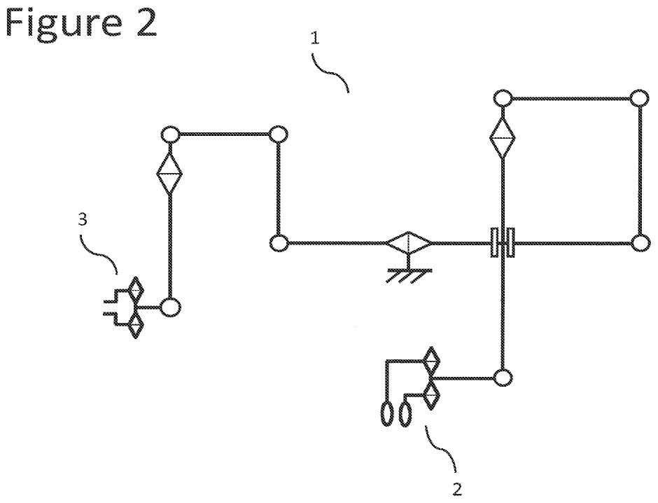

The sterile interface for articulated surgical instruments, according to an embodiment of the present invention, is intended to be used in a mechanical telemanipulator 1, like the one shown in FIG. 1, whose kinematic model is shown in FIG. 2. One of the key features of this kind of mechanical telemanipulator 1 lies in a master-slave architecture and mechanical transmission system, which enable a natural replication of the user hand movements on a proximal handle 2, by the end-effector 3 of a distal surgical instrument 4 on a remote location.

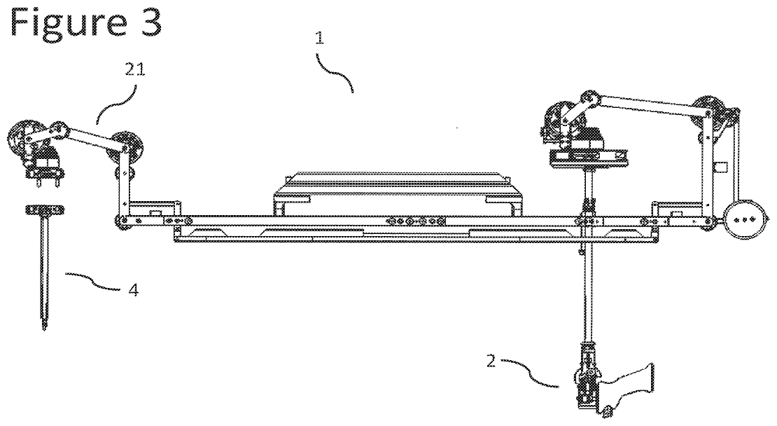

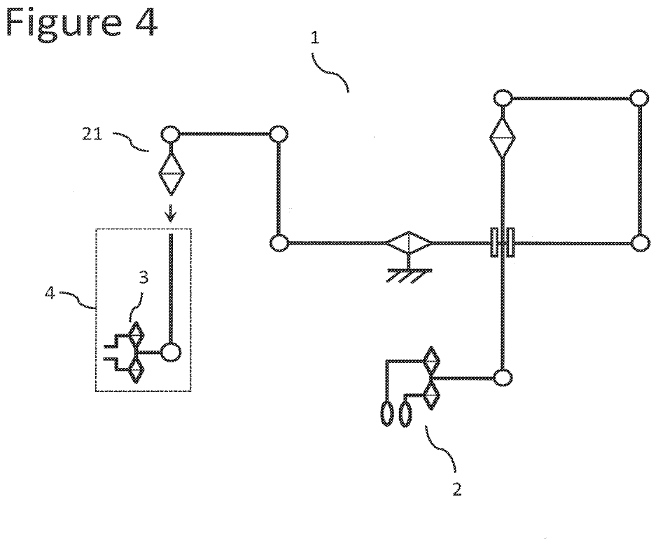

The surgical instrument 4 can take different functions and forms, like a dissector, scissor or grasper and can be plugged and unplugged from the mechanical telemanipulator 1 several times during the same surgical procedure (FIGS. 3 and 4). The remaining part of the mechanical telemanipulator 1, excluding the surgical instrument 4, is referred as the surgical platform 21. It is desirable for the surgical instruments being plugged and unplugged to be sterile while the surgical platform is non-sterile. These plugging/unplugging procedures involve not only the structural attachment/detachment of the proximal part of surgical instrument 4 to the distal part of the surgical platform 21 but also the connection/disconnection of the mechanical transmission systems that deliver motion from the different articulations of the proximal handle 2 to the equivalent articulations of the end-effector 3. In addition, these plugging/unplugging procedures have to be easily and quickly performed by the surgeons during the surgical procedure in order to avoid long and frustrating breaks in the surgeon's workflow.

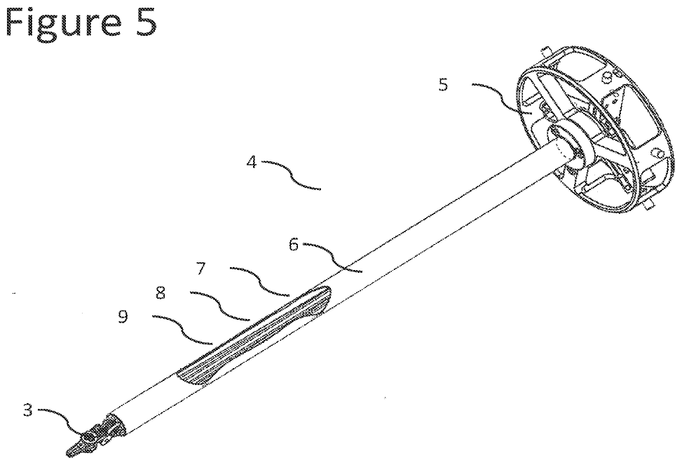

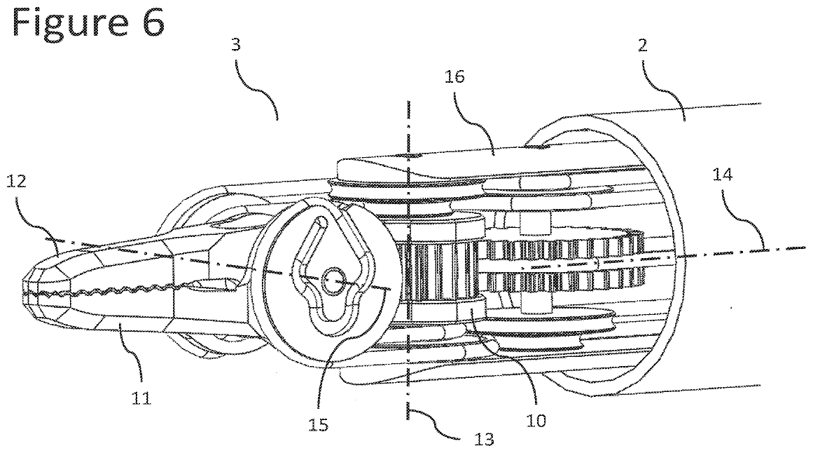

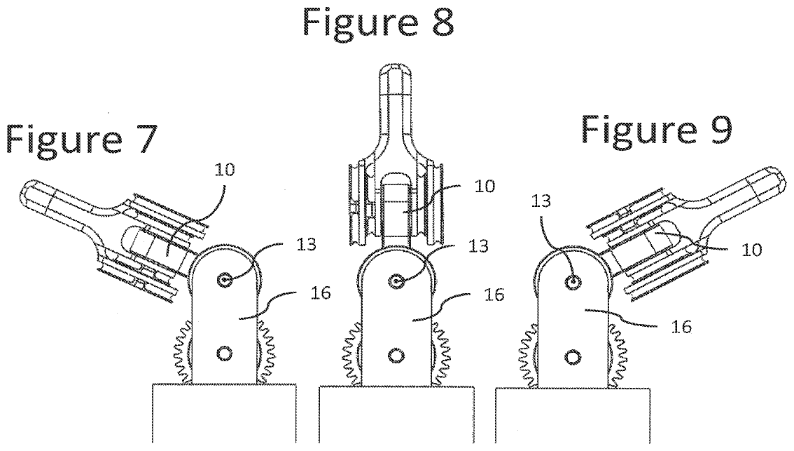

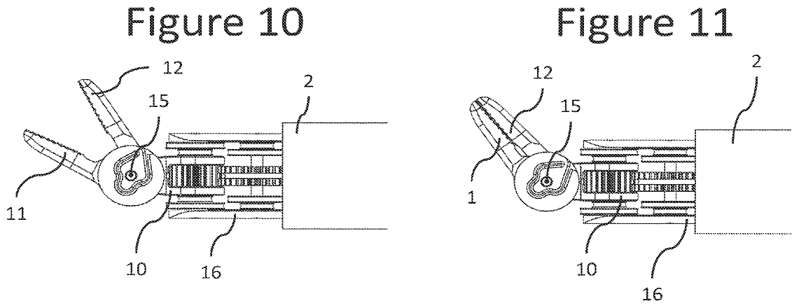

A surgical instrument 4 for minimally invasive surgical procedures, being able to be connected to an embodiment of the sterile surgical interface of the present invention, is described herein, and is seen generally in FIG. 5. This surgical instrument 4 includes a distal articulated end-effector 3, a proximal hub 5 and a main shaft 6, through which different mechanical elements 7, 8, 9 may pass, delivering motion to the different end-effector links 10, 11, 12 (FIG. 6) from the proximal hub 5. Referring to FIG. 6, the end-effector 3 is connected to the distal extremity of the main shaft 6 by a proximal joint, which allows the rotation of the proximal end-effector link 10 by the proximal axis 13 in such a manner that the orientation of the proximal end-effector link 10 with respect to the main shaft axis 14 can be changed. The distal end-effector links 11, 12 are pivotally connected to the proximal end-effector link 10 by two distal joints, having coincident axes of rotation, which are represented by the distal axis 15. This distal axis 15 is substantially perpendicular and non-intersecting with the proximal axis 13 and substantially intersects the main shaft axis 14. FIGS. 7 to 11 show the surgical instrument 4 with different angular displacements at the end-effector joints.

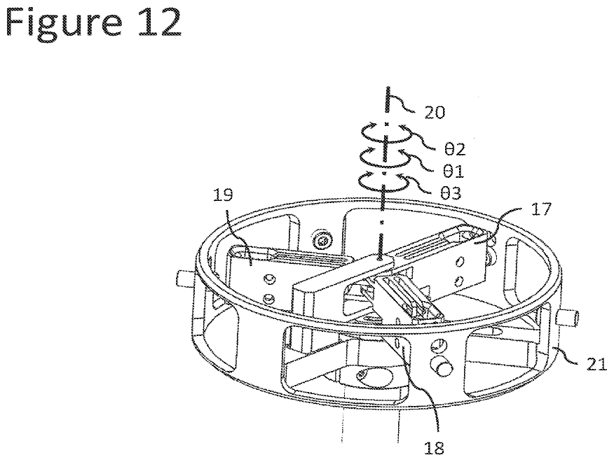

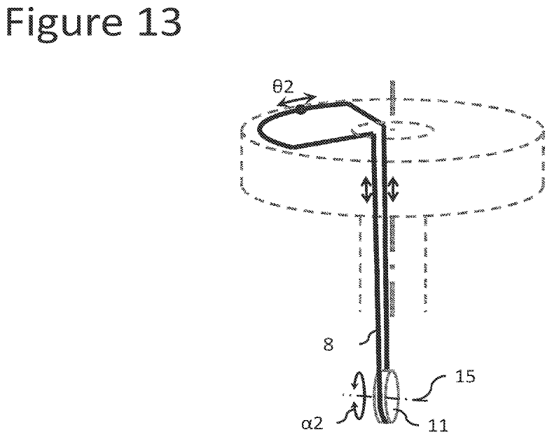

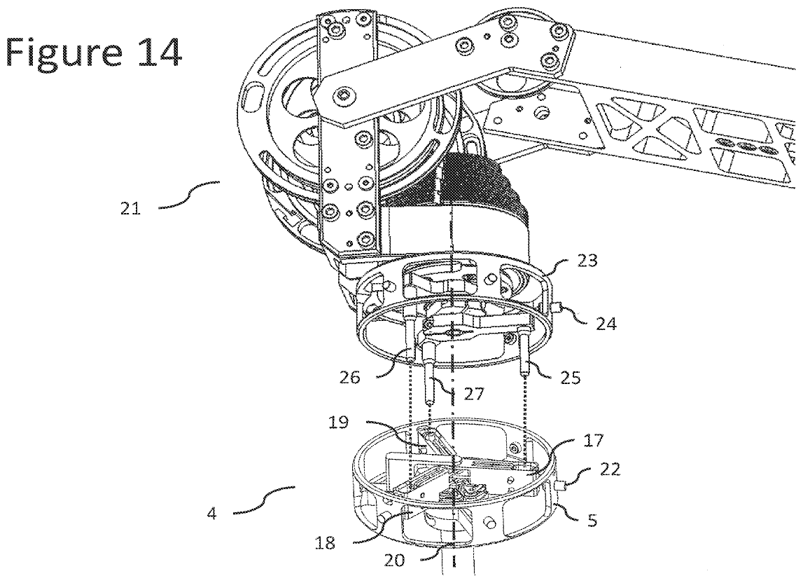

With reference to FIGS. 12 and 13, the movement is transmitted to each one of the three distal articulations of the instrument 4 by a rotating element 17, 18, 19, which is able to rotate about an axis 20 and is connected to a transmission element 7, 8, 9. As a result, when the rotating element 17, 18, 19 rotates a certain angle θ1, θ2, θ3 about the axis 20, a rotation α1, α2, α3 is transmitted to the respective end-effector link 10, 11, 12. Accordingly, FIG. 14 shows how the movement is transmitted to the rotating elements 17, 18, 19 of the surgical instrument 4 from the distal part of the surgical platform 21. The cylindrical elements 25, 26, 27, which are mounted inside the housing element 23, are able to translate along circular paths that are collinear with the axis 20. When the proximal hub 5 is attached to the housing element 23, the cylindrical elements 25, 26, 27 can be respectively connected to the rotating elements 17, 18, 19, so that the movements generated at the handle 2 can be transmitted to the three end-effector links 10, 11, 12 by the transmission elements 7, 8, 9.

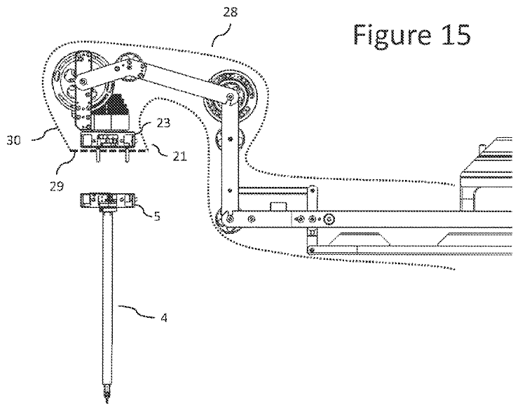

Since the surgical instrument 4 is entering the patient's body, it has to be sterile, just like the area in the vicinity of the patient. On the other hand, the surgical platform 21 is not sterile (and it is not desirable to have the entire surgical platform be part of the sterile field as this would not be practical in view of normal operating room workflow) and therefore should be separated from the sterile instrument portions by the sterile interface 28, which protects the sterile area from the non-sterile components of the surgical platform 21 (FIG. 15).

The sterile interface 28 comprises two main components: a flexible sleeve 30, which covers the moving links of the surgical platform 21 and a rigid connector 29, which i) guarantees that the sterile instrument 4 is not directly touching non-sterile components, ii) enables attachment/detachment between the surgical instrument 4 and the surgical platform 21, and iii) ensures the connection/disconnection of the mechanical transmission systems that deliver motion to the end-effector links 10, 11, 12. Full connection of the mechanical transmission systems during operation of the platform is necessary for faithful replication of operator hand movements at the end effector.

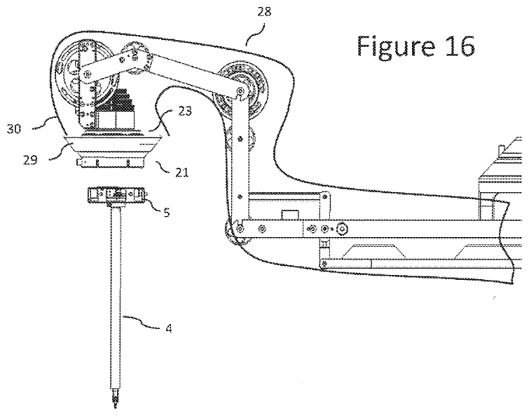

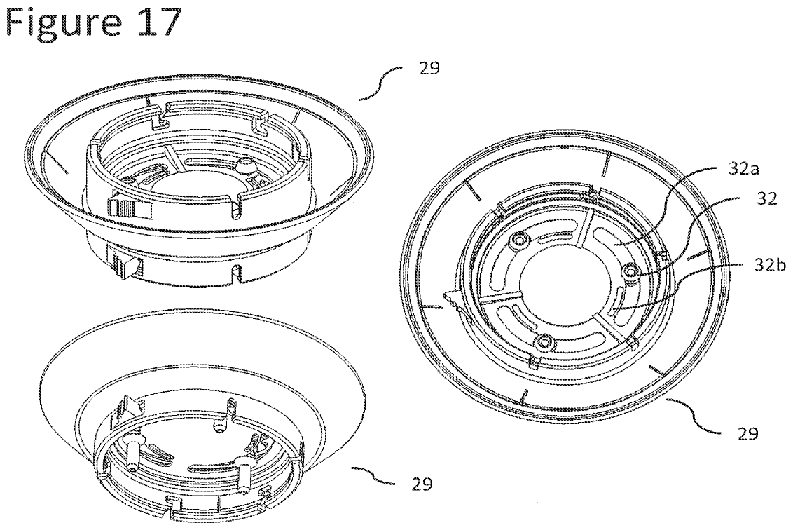

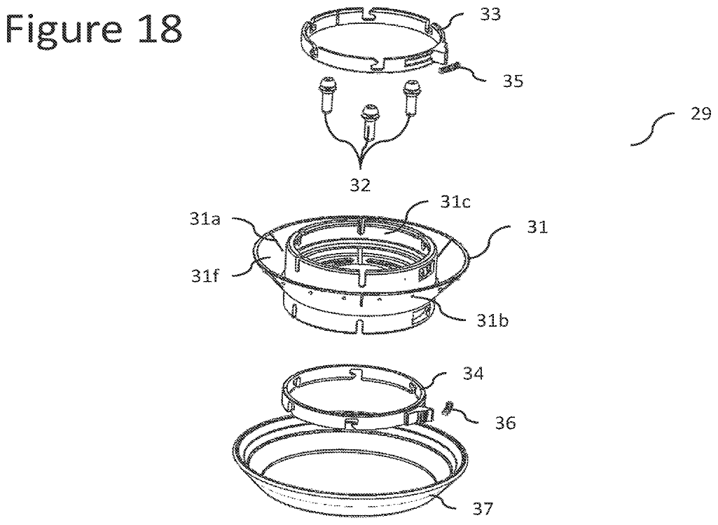

FIG. 16 shows an embodiment of the current invention where the sterile interface 28 comprises a plastic flexible sleeve 30 and a multi-component plastic rigid connector 29. This plastic rigid connector 29 can be either sterilisable/reprocessable or single-use. However, in this particular embodiment, it is considered to be single-use, just like the plastic flexible sleeve 30. FIGS. 17 and 18 show different 3D views of the rigid connector 29, with its multiple components 31, 32, 33, 34, 35, 36, 37. The three miniature cups 32 are able to move along three circular grooves 32a, where they are inserted at the level of the insertion grooves 32b. The core component 31 has two surfaces 31c where the rings 33 and 34 can rotate, actuated by the compression springs 35 and 36. The fixation ring 37 can be attached to the core component 31 by the deformation of the flanged surface 31f where the grooves 31a and the sharp points 31b are located.

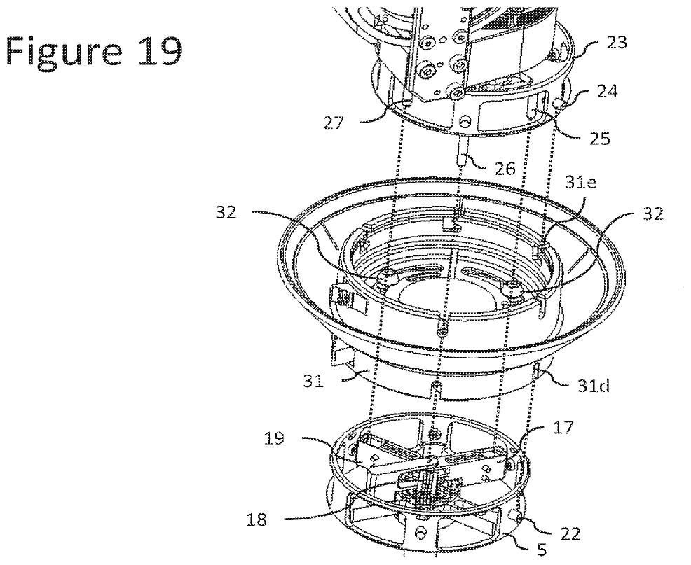

FIG. 19 shows how the rigid connector 29 can be positioned and operationally connected between the proximal hub 5 of the surgical instrument 4 and the housing element 23 of the surgical platform 21. In order to connect/disconnect the mechanical transmission systems that deliver motion to the end-effector links 10, 11, 12 the cylindrical elements 25, 26, 27 are inserted on the three miniature cups 32, which are then inserted on the rotating elements 17, 18, 19. In this way, it can be guaranteed that the sterile surgical instrument 4 is not directly touching non-sterile components. Since the rigid connector 29 can be a single-use product, its manufacturing processes have to guarantee fairly low production costs, which typically cannot deliver very accurate components. Therefore, by transmitting the movement, through the miniature cups 32, with translations on a maximized-diameter-circular path, this interface is less sensitive to dimensional inaccuracies or backlash between matching components. This is an improvement over other known devices where movement is transmitted by rotations with reduced diameters. A further advantage of this interface 28 pertains to its axisymmetric geometry, which is volumetrically optimized for rotations about the main shaft axis 14.

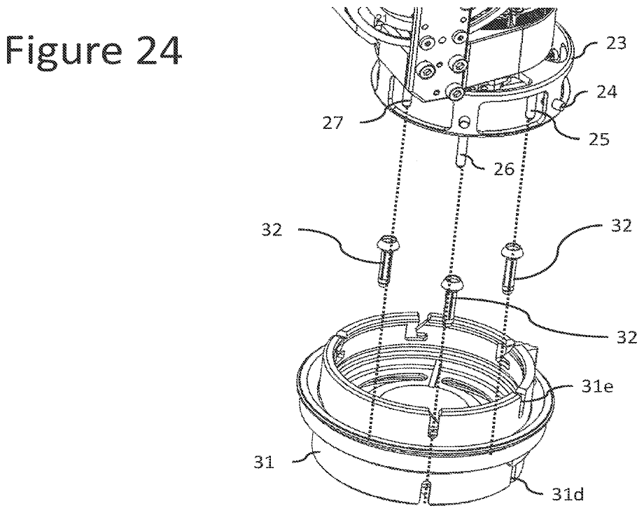

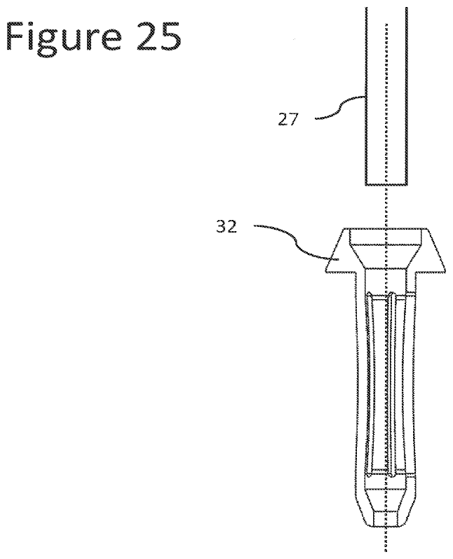

In another embodiment of the current invention (FIG. 24), the miniature cups 32 don't need to be pre-inserted in the three circular grooves 32a. Instead, they have a geometry which enables them to be pre-inserted directly on the cylindrical elements 25, 26, 27 before the attachment of the core element 31 on the housing element 23. As shown in FIG. 25, the miniature cups 32 can be attached directly to the cylindrical elements 25, 26, 27 thanks to their geometry, which comprises multiple longitudinal groves that enable the miniature cups to expand radially when the cylindrical elements 25, 26, 27 are inserted. Other solutions for the attachment of the miniature cups 32 on the cylindrical elements 25, 26, 27 can be used, using deformable components (like the one shown in FIG. 25) or non-deformable components (for instance, using threaded surfaces, the miniature cups 32 can be screwed on the cylindrical elements 25, 26, 27, or using magnets).

The structural attachment/detachment between the surgical instrument 4 and the remaining part of the surgical platform 21 is made by inserting the five radially-displaced platform pins 24 in the five radially-displaced connector grooves 31e. On the surgical instrument 4 side, the five radially-displaced instrument pins 22 are inserted in the five radially-displaced connector grooves 31d. As can be seen in FIG. 19, these two attachment mechanisms, used to attach the rigid connector 29 on the surgical platform 21 and the surgical instrument 4 on the rigid connector 29, have axi-asymmetric features or geometries (in the current embodiment, axi-asymmetric placement of radially-displaced pins and connector grooves) that prevent users from inserting the sterile articulated instruments on a wrong axial direction.

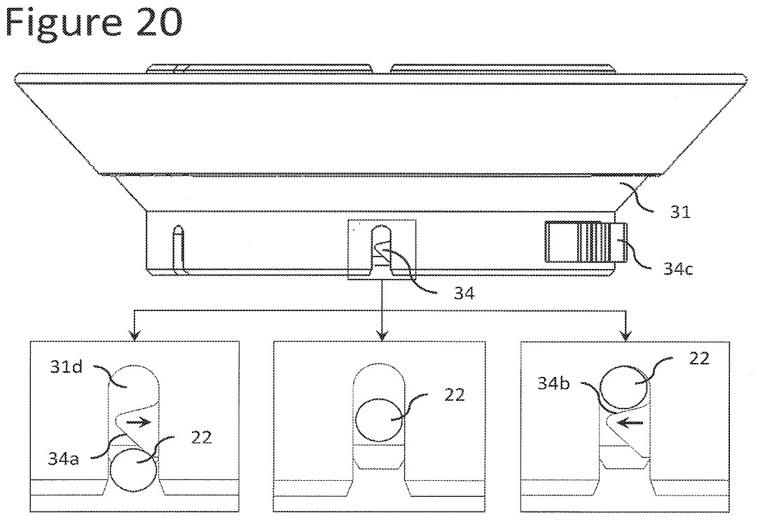

FIG. 20 shows in detail the attachment mechanism between each instrument pin 22 and the respective connector groove 31d. When the instrument pin 22 enters the connector groove 31d, it touches the angular surface 34a of the ring 34, causing its angular displacement against the compression spring 36. This angular displacement allows the instrument pin 22 to reach the end of the connector groove 31d, where it is kept in place by the action of the compression spring 36, whose force presses the angular surface 34b of the ring 34 against the instrument pin 22. This sequence is simultaneously done at all the radially-displaced instrument pins 22, guarantecing the structural attachment between the surgical instrument 4 and the rigid connector 29. The structural detachment between the surgical instrument 4 and the rigid connector 29 is achieved by the reverse sequence of actions. The structural attachment/detachment between the rigid connector 29 and the housing element 23 of the surgical platform 21 is performed in a similar interaction between each platform pin 24 and its respective connector groove 31e.

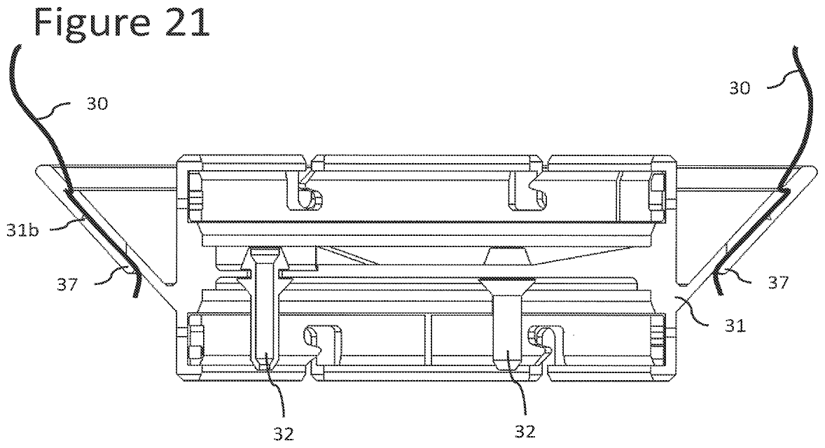

FIG. 21 shows how the flexible sleeve 30 can be releasably attached to the rigid connector 29, by being squeezed between the flanged surface 31f of the core component 31 and the fixation ring 37. The indentation of the sharp points 31b on the flexible sleeve 30 reinforces the attachment. This method of attachment is an improvement over prior art interfaces where the flexible sleeve 30 is glued or welded to the rigid connector 29, which jeopardizes the possibility of having the flexible sleeve 30 as a single-use product and the rigid connector 29 as a reusable device. Therefore, with this feature in the interface as per the current invention, the rigid connector 29 can be cleaned and sterilized after each procedure, which can significantly reduce procedure costs over the use of prior art solutions.

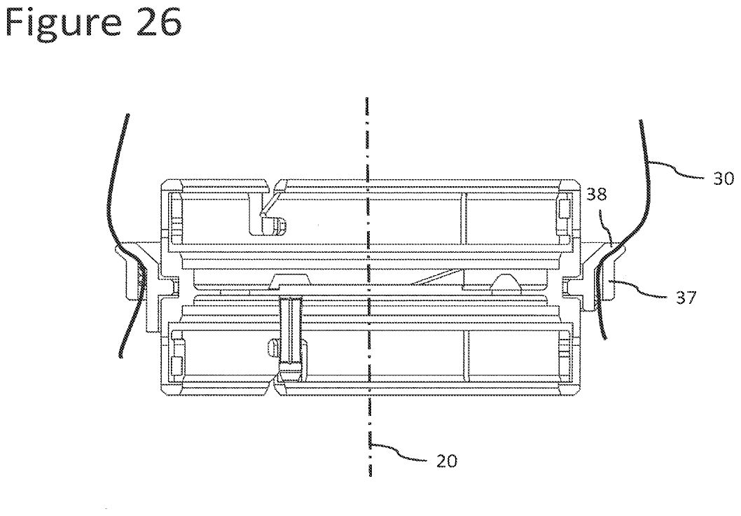

In another embodiment of the current invention (FIG. 26), the fixation ring 37 may be fixed to a rotating ring 38, which is able to freely rotate around the core component 31. In this embodiment, the flexible sleeve 30 is squeezed between the fixation ring 37 and the rotating ring 38 and its torsional deformation is minimized when the core component 31 is rotated around the axis 20 by the platform 21.

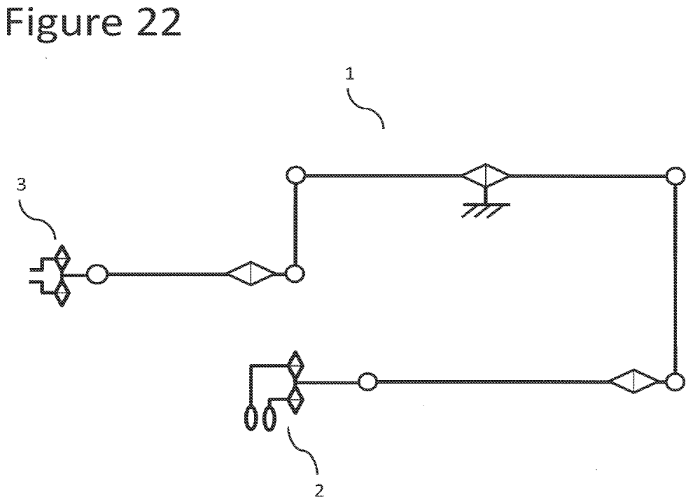

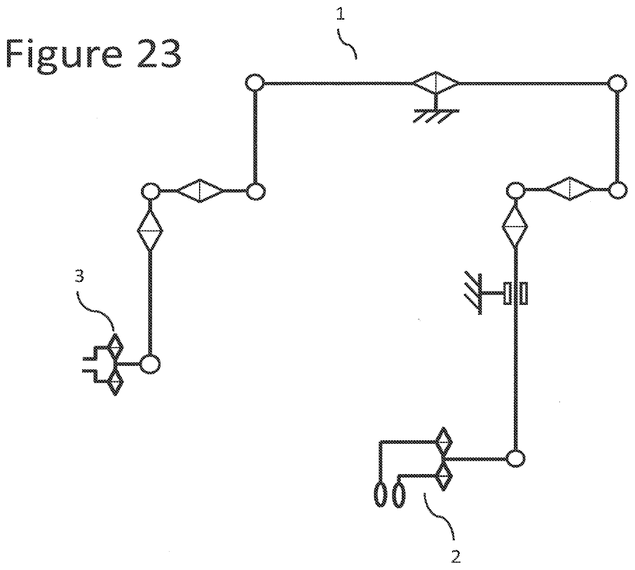

While this invention has been shown and described with reference to particular embodiments thereof, it will be understood by those skilled in the art that various changes in form and details may be made therein without departing from the spirit and scope of the invention as defined by the appended claims. For instance, the mechanical telemanipulator 1 can assume other kinematics, like the ones shown in FIGS. 22 and 23. In addition, while the sterile interface of the present invention has been primarily described in connection with a laparoscopic surgical platform, one of skill in the art will understand that the sterile interface could easily be used with other surgical platforms, such as open field systems. In addition, the current sterile interface could be used with other telemanipulator or remote actuation systems in other sterile situations outside of the surgical context.

1. A method for maintaining sterility of surgical instruments during a surgical procedure, the method comprising:
locking a rigid connector to a surgical platform;
locking the rigid connector to a proximal hub of an articulated surgical instrument;
covering moving links of the surgical platform with a flexible sleeve coupled to the rigid connector;
moving an end-effector of the articulated surgical instrument responsive to movement at the surgical platform to perform the surgical procedure while maintaining sterility of the articulated surgical instrument during the surgical procedure;
detaching the articulated surgical instrument from the rigid connector; and
removing the flexible sleeve from the surgical platform.

2. The method of claim 1, wherein covering moving links of the surgical platform via the flexible sleeve ensures that the articulated surgical instrument is not directly touching the surgical platform during the surgical procedure.

3. The method of claim 1, further comprising mating at least one miniature cup with both a mechanical transmission from the surgical platform and a mechanical transmission of the articulated surgical instrument.

4. The method of claim 1, wherein removing the flexible sleeve from the surgical platform comprises detaching the flexible sleeve from the rigid connector.

5. The method of claim 1, further comprising attaching and detaching the articulated surgical instrument from the surgical platform several times during the surgical procedure.

6. The method of claim 1, further comprising sterilizing the rigid connector after each surgical procedure.
